(19) United States
(12) Patent Application Publication
(10) Pub. No.: US 2011/0098787 A1
(43) Pub. Date: Apr. 28, 2011

(54) COCHLEAR IMPLANT

(76) Inventors: Michael R. Spearman, The Woodlands, TX (US); Brian M. Spearman, The Woodlands, TX (US)

(21) Appl. No.: 12/914,618

(22) Filed: Oct. 28, 2010

Related U.S. Application Data
(60) Provisional application No. 61/255,562, filed on Oct. 28, 2009.

(51) Int. Cl. A61F 11/04; A61N 1/36

(52) U.S. Cl. 607/57

(57) ABSTRACT

An improved cochlear implant and method are disclosed. The present invention relates particularly to an external processing unit of a cochlear implant which processes sounds from within the ear canal of the patient. The implant comprises a microphone placed within the ear canal of the patient. The implant can alternatively comprise a hollow tube placed within the ear canal of the patient which is used to carry sound to a microphone located elsewhere. The method may comprise the steps of: providing a microphone in the ear canal, thereby improving the hearing of said hearing-impaired subject. The method may comprise the steps of: providing in the ear canal a tube that delivers sound to a microphone, thereby improving the hearing of said hearing-impaired subject.

### RELATED APPLICATIONS

This application claims the benefit under 35 U.S.C. 119(e) of U.S. Provisional Patent Application No. 61/255,562, filed on Oct. 28, 2009 under the provisions of 35 U.S.C. 111(b) The contents of this provisional application are fully incorporated herein by reference.

### FIELD OF THE INVENTION

The present invention relates to generally to the field of cochlear implants and improvements in obtaining sound quality for cochlear implant patients. The present invention relates more particularly to an external processing unit of a cochlear implant which processes sounds from within the ear canal of the patient. The present invention relates most particularly to the improved placement of the microphone used with cochlear implants to take advantage of the Pinna Effect and the Ear Canal Resonance of the human ear.

### BACKGROUND OF THE INVENTION

Cochlear implants are used to help the profoundly deaf to hear. However, a major problem for cochlear implant users is the difficulty to hear speech in noisy environments such as restaurants and groups of people.

Cochlear implants typically consist of two main parts, an internal receiver unit and an external processor unit. The internal unit is surgically implanted posterior to, or behind, the pinna of the ear of the patient. The internal unit has at least one electrode that is inserted into the cochlea. The internal unit also includes an internal coil assembly which receives signals from the external unit which then are carried to the electrode and into the cochlea. The internal unit also includes a magnet which is used to hold the external unit against the skin on the outside of the head.

The external unit also includes a microphone and signal processing component, and an external coil assembly. The external unit also includes a magnet for suspending the external unit utilizing the field of the magnet contained in the internal unit. These are located on the mastoid region behind the ear of the patient.

Alternatively, the external unit, can be in the form of a behind-the-ear (BTE) configuration (similar to a hearing aid) with the external coil attached to the BTE by an electric wire. The magnet is contained in the external coil assembly to hold the coil against the skin on the outside of the head. The BTE is held in place on the ear.

Audio sounds are picked up by the microphone of the external unit and converted into electrical signals. The electrical signals are then processed by the signal processor of the external unit and then transmitted across the skin to the implanted internal processor/receiver unit. The electrical signals from the internal unit are carried to the electrode and into the cochlea. It is this electric current which directly stimulates the auditory nerve and provides the user with the sensation of hearing.

Usually, the microphone in the external units is located either on top of, or behind, the pinna of the ear. There are several problems with this placement.

Microphones pick up all sounds presented to the patient without any filtering of background noise or amplification of speech. As a result, the processor is required to attempt to attempt do this difficult task. The best processors use noise cancellation techniques to cancel low frequency background sounds and directional microphones to create a “cone of hearing” in the direction that the patient is looking while talking to the person they are speaking with. While this is somewhat effective, it is not the methodology that normal people use in distinguishing speech from noise and hence, not nearly as effective.

Whether in a mastoid or BTE configuration, cochlear implant patients have problems with external noises such as wind and movement of hair, glasses and hats since the microphones of the external processors are relatively exposed to the environment.

The location of the microphones also present problems for patients who attempt to use telephones. U.S. Pat. No. 7,167,572 (Harrison et al.) sought to improve upon this by providing a microphone which is placed within the concha of the ear. When a telephone handset is held against the ear, the phone seals against the outer ear creating a chamber wherein the microphone resides. This improves the acoustic response of a BTE system during telephone use. However, while placing the microphone in the concha aids in telephone use, it does little to help cochlear implant patients in noisy environments.

People with normal hearing hear very well in noisy environments and in groups of people. While background noise is present, they are able to hear voices quite well. The reason for this is the effect on sound of the natural anatomy of the ear including the outer ear, or pinna, and the ear canal. The pinna gathers and directs sound to the ear canal. It is especially effective in mid and high frequencies (up to 10-15 dB or 3 times increase in loudness).

The ear canal provides natural amplification by functioning as a resonate tube. The canal resonance effect can be determined by the wavelength of sound and the geometry of tube match. The formula for resonance within a tube is F=V/(4L) where V=the speed of sound (velocity)=344 m/s, and L is the length of the tube in meters. Since an ear canal is approximately equal to 25 mm, or 0.025 m, F=344/(4*0.025)=3440 Hz. When calculated over the audible sound frequency spectrum for typical ear canals (see FIG. 4), one sees that the ear canal is very effective at providing an increase in amplification in the mid to high frequencies (up to 10-15 dB or additional 3 times increase in loudness). This is very important as the mid to high frequencies are critical frequencies for discerning speech.

The effect of the pinna and canal resonance together are additive. FIG. 4 shows the pinna effect, ear canal resonance and total resulting effects. Therefore, what is needed is a device for cochlear implant patients which is able to take advantage of these effects and provide cochlear implant patients with better hearing in noisy environments.

### SUMMARY OF THE INVENTION

The present invention utilizes a cochlear implant with the microphone located deep within the ear canal so as to take advantage of the pinna effect and ear canal resonance effects. Such a placement of the microphone allows the sounds that the microphone picks up to be similar to those heard by a normal hearing person, thereby naturally amplifying the mid to high frequency speech sounds in relation to the low frequency background noise. This placement also eliminates environmental sounds such as wind noise, hair movement, etc. since the microphone is now concealed within the ear canal and sheltered from these. It also provides a much more effective and natural sound input to the processor, and better results for hearing speech over background noise.

The microphone may be located at any point within the ear canal but preferably as close to the tympanic membrane as possible to take more advantage of the resonance effect. Placement can typically be within 5 mm of the tympanic membrane. However, this may not be possible in some patients due to a difficult geometry of their canal and placement may be limited to 5 to 15 mm from the tympanic membrane. Even in these situations, the patient will derive substantial benefits from the pinna effect and some resonance, as well as having the reduction in wind noise, hair movement, etc.

The microphone placement in the canal may be used with either a mastoid or BTE style cochlear implant. An electric wire is connected from the cochlear implant processor to the microphone with sufficient length to place the microphone in the desired location within the ear canal. The microphone can be supported within the canal by the wire connected to the microphone if it is sufficiently stiff, or can be supported with means similar to supporting receivers in ear canals of hearing aid patients, such as a custom molded support structure, or a preformed, flexible support structure.

While the preferred method is to place the microphone in the canal, an alternate method is to place the microphone in the processor unit, with a flexible tube connected to it which is long enough to be formed and placed within the ear canal to gather sounds from within the ear canal and deliver them to the microphone. While there may be a slight attenuation (approximately 3-4 dB) of some frequencies within the tubing, this can be very useful for small diameter or difficult geometry ear canals for placing the point of sound pickup as close to the tympanic membrane as possible with the benefits still being far greater than the typical external microphone configuration.

The present invention may also include two or more microphones located within the ear canal. It may be used with cochlear implants with digital or analog processors, single or multiple electrodes, and single or multiple channels.

It should be readily apparent to those skilled in the art that this improved device may be used on any cochlear implant configuration for patients with profound hearing loss to improve their ability to understand speech against noise backgrounds and better hearing in general.

### BRIEF DESCRIPTION OF THE DRAWINGS

The above, as well as other advantages of the present invention, will become readily apparent to those skilled in the art from the following detailed description when considered in the light of the accompanying drawings in which like numerals designate corresponding parts in the several views.

FIG. 1 is an environmental view of a prior art device as described in Harrison et al. (U.S. Pat. No. 7,167,572) showing a global view of the outer ear, ear canal, and inner ear in cross-section, with the microphone of the external processor placed in the concha of the outer ear, but not within the ear canal.

FIG. 2 is an environmental view of an embodiment of the present invention showing a global view of the outer ear, ear canal, and inner ear in cross-section, with the microphone of the present invention placed in the ear canal.

FIG. 3 is an environmental view of an alternative embodiment of the present invention showing a global view of the outer ear, ear canal, and inner ear in cross-section, with a tube of the present invention placed in the ear canal to carry sound to the microphone of the external processor.

FIG. 4 is a graph depicting the effect on sound amplification provided by the pinna effect and ear canal resonance.

FIG. 5 is an illustration of a molded support structure which holds the microphone or tube in place in the ear canal.

FIG. 6 is an illustration of an alternative molded support structure which holds the microphone or tube in place in the ear canal.

### DETAILED DESCRIPTION OF THE PREFERRED EMBODIMENTS

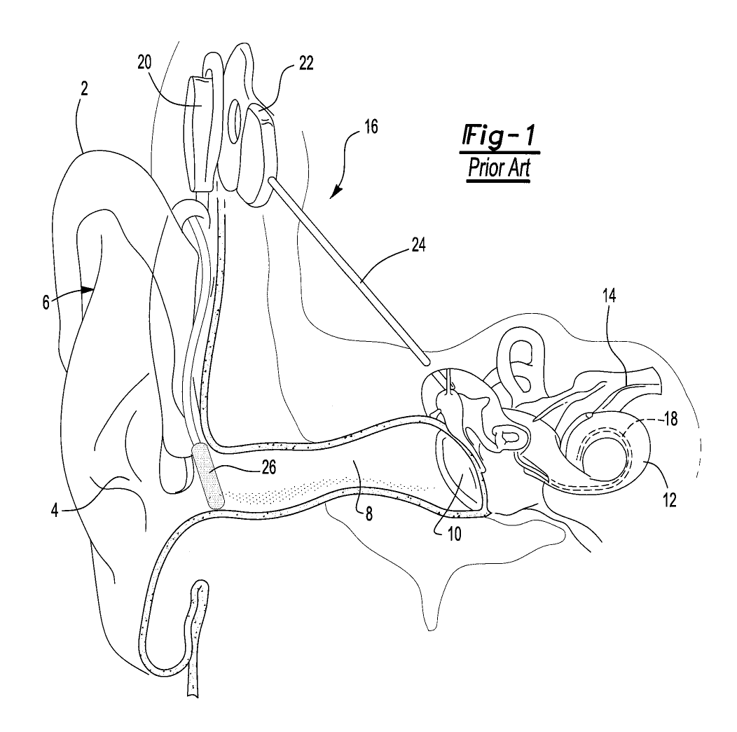

FIG. 1 shows an attempt in the prior art to improve the hearing of cochlear implant patients. Illustrated is the pinna 2, concha 4, and outer ear 6 of the human ear, as well as the ear canal 8, tympanic membrane (ear drum) 10, cochlea 12 and auditory nerve 14. Cochlear implant 16 comprising microphone 26, external processor 20, internal processor 22, connection 20 and implanted electrode 22 are also shown. The prior art has improved the hearing of cochlear implant patients by placing a microphone 26 just outside the ear canal 8 of the patient in the concha 4 of the patients a ear. As stated herein above, this provides for the use of a telephone by the cochlear implant patient. However, it does not take into account the pinna effect or ear canal resonance described above.

FIG. 2 illustrates one embodiment of the present invention, wherein a microphone 26, which may be the same as the microphone used in prior art devices, but may also be any microphone which is suitable for use with cochlear implant devices, such as a single or dual microphone, is connected at a first end 28A of a segment of flexible tubing or wire 28 placed in the ear canal 8. The second, or other, end 28B of the flexible tubing or wire 28 is connected to the external processor 20 by means known in the art. The flexible tubing or wire 28 may be stiff enough to hold the microphone 26 in the ear canal 8, or it may be held in place by a support structure 32 which may be molded or formed from any suitable material known in the art. The flexible tubing or wire 28 may be electrically conductive to carry a signal from the microphone 26 to the external processor 20, or a separate wire (not shown) may be used. While the various figures show support structure (32) actually holding up the microphone (26), it is well within the scope of the present invention that the support structure 32 could be placed behind the microphone 26 to support the flexible tubing or wire 28.

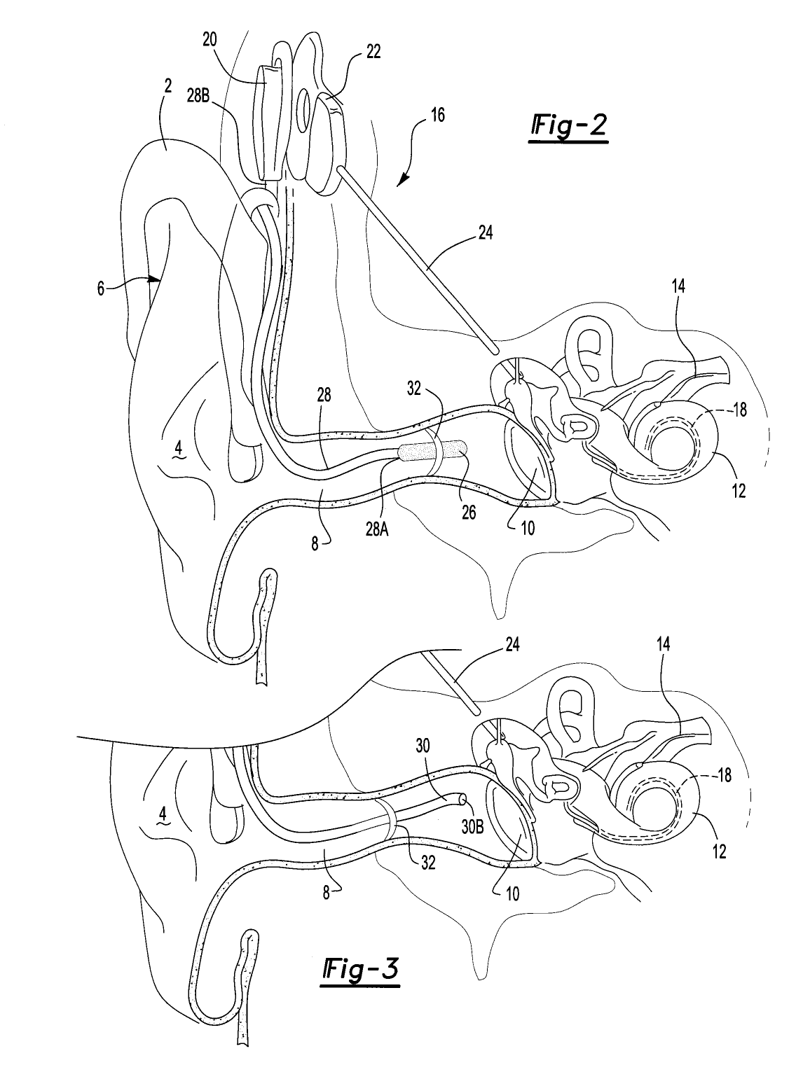

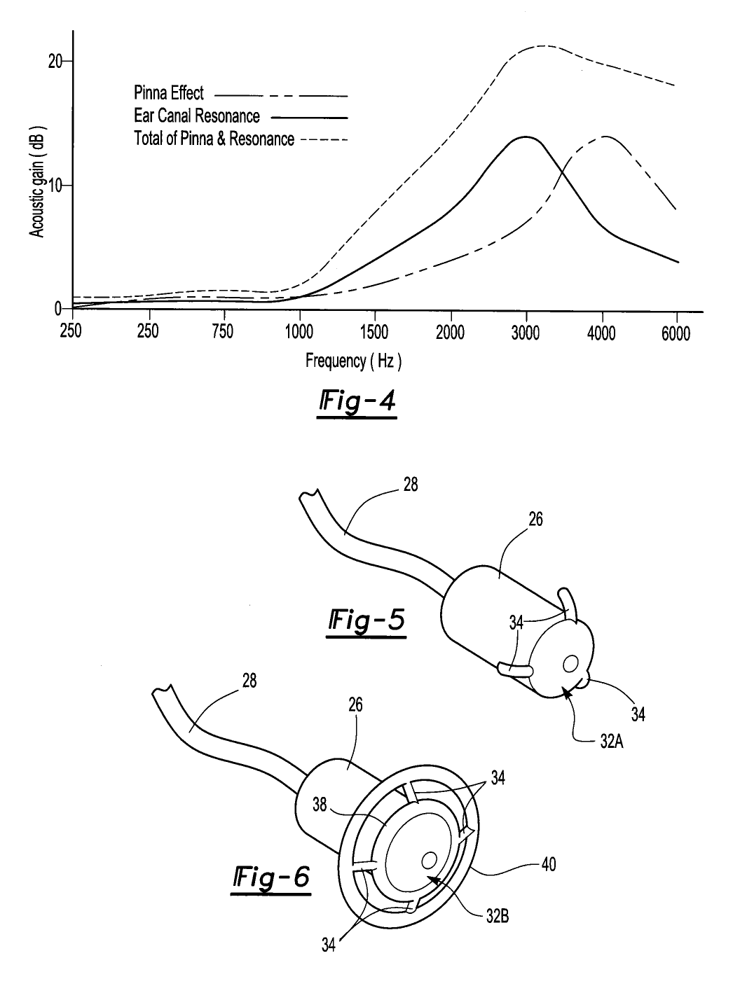

FIG. 3 Shows another embodiment of the present invention, which utilizes a segment of flexible hollow tubing 30 placed in the ear canal 8 for the purpose of carrying sound to the external processor An end 30A of the flexible hollow tubing 30 is secured to the support structure 32, while the other end (not shown) is secured to the external processor 20 (see FIG. 2).

FIG. 5 shows one preferred configuration for the support structure 32 used to hold the microphone (26) in place. The spoke-type support structure 32A contains spokes 34 anchored to a central portion (36) of the spoke-type support structure 32A. The spoke-type support structure can also be used to hold the flexible tubing or wire 28, or flexible hollow tubing 30 in place.

FIG. 6 shows another preferred configuration of a support structure 32 in the form of a ring-type support structure 32B used to hold the microphone 26 in place. The ring-type support structure 32B contains spokes (34) anchored both to an outer rim or ring 38 and an inner rim or ring 40, much like the ‘basket’ of a ski pole. The ring type support structure 32B is then attached to the microphone 26 by any suitable means. The ring type support structure 32B may also be used to support the flexible tubing or wire 28, or flexible hollow tubing (30) in place. Other configurations of support structures 32 are well within the scope of the present invention.

The present invention can be used with any external processor which can be used with a cochlear implant. The microphone can be of any suitable type for use with cochlear implants, which include, but are not limited to single or dual microphones.

The flexible tubing or wire 28 can be made of any material which is suitable for use with cochlear implant devices, and should be of sufficient length to hold the microphone as close to the ear drum or tympanic membrane as possible, such as in a range of 2-20 mm from the eardrum, but preferably 2 mm.

Alternatively, when microphone 26 is located in the external processor 20, the flexible hollow tubing 30 can be made of any material suitable for use with cochlear implant devices, and the open or free end 30A of the hollow tube 30 should be of sufficient length to be placed as close to the ear drum or tympanic membrane as possible, such as in a range of 2-20 mm from the eardrum, but preferably 2 mm.

In accordance with the provisions of the patent statutes, the present invention has been described in what is considered to represent its preferred embodiments. However, it should be noted that the invention can be practiced otherwise than as specifically illustrated and described without departing from its spirit or scope.

What is claimed is:

1. A cochlear implant for patients with profound hearing loss comprising:
a) an external processor unit which includes a speech processor;
b) a microphone located within the ear canal of the patient being isolated from the processor unit, and being operatively connected to the processor unit.

2. The device in claim 1 wherein the microphone is located within 2 mm of the tympanic membrane.

3. The device in claim 1 wherein the microphone is located within 10 mm of the tympanic membrane.

4. The device in claim 1 wherein the microphone is located within 15 mm of the tympanic membrane.

5. The device of claim 1 wherein the microphone is located within 20 mm of the tympanic membrane.

6. The device of claim 1, wherein the microphone is located within a range of 2-20 mm of the tympanic membrane.

7. A cochlear implant for patients with profound hearing loss comprising:
a) an external processor unit which includes a speech processor and a microphone located outside of the ear canal; and
b) a hollow tube positioned within the ear canal wherein the ear canal tube has an open end in the ear canal for receiving sound and the opposite end is connected to the input of the microphone.

8. The device in claim 7 wherein the open end of the tube is located within 2 mm of the tympanic membrane.

9. The device in claim 7 wherein the open end of the tube is located within 10 mm of the tympanic membrane.

10. The device in claim 7 wherein the open end of the tube is located within 15 mm of the tympanic membrane.

11. The device in claim 7 wherein the open end of the tube is located within 20 mm of the tympanic membrane.

12. The device of claim 7, wherein the microphone is located within a range of 2-20 mm of the tympanic membrane.

13. A method for improving the hearing of a hearing-impaired subject, said method comprising the step of: providing a microphone in the ear canal, thereby improving the hearing of said hearing-impaired subject.

14. A method for improving the hearing of a hearing-impaired subject, said method comprising the step of: providing in the ear canal of the hearing impaired subject within 2-20 mm of the tympanic membrane, a tube that delivers sound to a microphone, thereby improving the hearing of said hearing-impaired subject.

15. A method for improving the hearing of a hearing-impaired subject, said method comprising the steps of:
providing a microphone in the ear canal of the hearing impaired subject within 2-20 mm of the tympanic membrane, and
connecting the microphone to an external processor unit of a cochlear implant device worn by the hearing impaired patient,
thereby improving the hearing of said hearing-impaired subject.

16. A method for improving the hearing of a hearing-impaired subject, said method comprising the step of:
providing a microphone in the ear canal, within a range of 2-20 mm of the tympanic membrane, of the hearing impaired subject,
thereby improving the hearing of said hearing-impaired subject.

17. A method for improving the hearing of a hearing-impaired subject, said method comprising the step of:
providing a microphone in the ear canal, thereby picking up sound as amplified by the pinna effect and ear canal resonance, thereby improving the hearing of said hearing-impaired subject.

18. A method for improving the hearing of a hearing-impaired subject, said method comprising the steps of:
providing a microphone in the ear canal, and
connecting the microphone to an external processor unit,
said external processor unit delivering a signal to an internal processor unit implanted within the cochlea
thereby improving the hearing of said hearing-impaired subject.
